(19) United States
(12) Patent Application Publication
(10) Pub. No.: US 2023/0043649 A1
(43) Pub. Date: Feb. 9, 2023

(54) INFUSION PROTECTION AND SECURING MECHANISM

(71) Applicant: April Crawford, Oliver Springs, TN (US)

(72) Inventor: April Crawford, Oliver Springs, TN (US)

(21) Appl. No.: 17/393,763

(22) Filed: Aug. 4, 2021

(51) Int. Cl. A61M 5/14

(52) U.S. Cl. CPC A61M 5/1415; A61M 2005/1416; A61M 2210/083; A61M 2205/0216

(57) ABSTRACT

A protection apparatus for use with a continuous infusion fluid source and a peripheral fluid line is provided. The peripheral fluid line comprises an internal portion positioned within a vein located in a first appendage of a patient and an external lumen portion positioned on the exterior of the first appendage. The external lumen portion is configured to interface with a continuous infusion fluid source. The protection apparatus comprises a tubular sleeve of a first length configured to concentrically enclose the first appendage and the external lumen portion of the peripheral fluid line. The tubular sleeve may comprise a pouch portion located a distal end of the tubular sleeve, the pouch portion defining a volume configured to contain and secure the continuous infusion fluid source. The tubular sleeve may comprise a lumen window configured to enable physical and/or visual access to the external lumen portion of the peripheral fluid line.

### BACKGROUND

### Related Field

The embodiments described herein relate generally to medical equipment for personal use and, in particular, to a device or garment configured for use for and/or during continuous infusion of fluid or medication.

### Related Art

Treatment of various diseases may involve the continuous infusion of fluids and/or medication. For example, cancer patients may be continuously infused with chemotherapy medication, and individuals diagnosed with multiple sclerosis may be continuously infused with various disease-modifying drugs. Continuous infusion of medication involves directly administering the medication into the bloodstream of a patient, whereupon the medication acts accordingly to alleviate pain, minimize symptoms, cure a disease, and/or the like. In some instances, medication may be infused using a peripheral fluid line, such as a peripheral intravenous line and/or a peripherally inserted central catheter.

Due to the extended duration or span of continuous infusion of medications, a peripheral fluid line may remain implanted in a patient for long periods of time, while may be inconvenient for the patient. Specifically, the patient may be required to leave an exterior portion of the peripheral fluid line through which medication is administered undisturbed, while also maintaining a fluid connection or interface between the peripheral fluid line and the medication source (e.g., by remaining near the medication source at all times). Peripheral fluid lines may be further problematic by causing pain to the individual around the portion of the individual's body where the exterior portion of the peripheral fluid line is positioned, and such pain may be amplified when the exterior portion, a fluid connection or interface between the exterior portion and the medication source, and/or the medication source itself are physically disturbed or impacted.

### BRIEF SUMMARY

Various embodiments described herein are directed to a protection apparatus, device, or garment configured for use with a peripheral fluid line, such as a peripherally inserted central catheter (PICC), and a continuous infusion fluid source. In some embodiments, the continuous infusion fluid source is a balloon pump storing fluid medication and configured to administer the fluid medication to an adult person via the peripheral fluid line. In some embodiments, a protection apparatus includes a tubular sleeve with a pouch portion and is configured to secure a continuous infusion fluid source while maintaining a fluid connection between the continuous infusion fluid source and the peripheral fluid line. In some embodiments, the pouch portion is configured to secure the balloon pump. For example, the pouch portion comprises means for at least enabling the balloon pump to be inserted into the pouch portion and securing the balloon pump within the pouch portion. In some embodiments, the tubular sleeve is configured to be worn on an appendage of the patient on which the external lumen portion of the peripheral fluid line is positioned, such that the tubular sleeve covers the external lumen portion and the balloon pump is secured by the pouch portion, and comprises fabric materials.

In accordance with one aspect, a protection apparatus for use with a continuous infusion fluid source and a peripheral fluid line is provided. The peripheral fluid line includes an internal portion positioned within a vein located in a first appendage of a patient. The peripheral fluid line also includes an external lumen portion positioned on the exterior of the first appendage and configured to interface with a continuous infusion fluid source. The protection apparatus includes a tubular sleeve of a first length configured to concentrically enclose the first appendage and the external lumen portion of the peripheral fluid line. The tubular sleeve includes a pouch portion located at a distal end of the tubular sleeve, the pouch portion defining a volume configured to contain and secure the continuous infusion fluid source.

In various embodiments, the tubular sleeve includes a lumen window configured to enable physical and/or visual access to the external lumen portion of the peripheral fluid line. In various embodiments, the lumen window includes a flap configured to be opened to enable physical access to the external lumen portion of the peripheral fluid line. In various embodiments, the tubular sleeve includes a longitudinal channel spanning a distance from the pouch portion to the lumen window and configured to secure a fluid connection line connecting the continuous infusion fluid source and the external lumen portion of the peripheral fluid line.

In various embodiments, the tubular sleeve is configured to attach with and/or detach from an article of clothing worn by the patient. In various embodiments, the tubular sleeve is configured to attach with and/or detach from an additional tubular longitudinal portion such that the tubular sleeve is a second length different than the first length.

In various embodiments, the pouch portion circumferentially spans approximately half of the circumference of the tubular sleeve and is located on an anterior-facing aspect of the tubular sleeve. In various embodiments, the pouch portion circumferentially spans approximately half of the circumference of the tubular sleeve and is located on an anterior-facing aspect of the tubular sleeve. In various embodiments, the pouch portion and the lumen window are axially aligned along an axis substantially parallel to a longitudinal axis of the tubular sleeve. In various embodiments the tubular sleeve is ambidextrous, and the pouch portion is co-located with a posterior-facing or dorsal aspect of a second appendage. In various embodiments, the pouch portion is composed of elastic material and is configured to expand to a state in which the continuous infusion fluid source may be inserted into the volume defined by the pouch portion. In various embodiments, the pouch portion includes an expansion mechanism configured to cause the pouch portion to expand to a state in which the continuous infusion fluid source may be inserted into the volume defined by the pouch portion. In various embodiments, the pouch portion includes a retention mechanism configured to immobilize the continuous infusion fluid source within the volume defined by the pouch portion.

In various embodiments, the appendage is an arm of the patient, and the volume defined by the pouch portion is substantially co-located with a palmar region of the hand of the arm while the tubular sleeve concentrically encloses the arm. In various embodiments, the tubular sleeve includes one or more openings through which one or more digits of the hand may protrude while the tubular sleeve concentrically encloses the arm. In various embodiments, the tubular sleeve is composed of elastic material. In various embodiments, a diameter of the tubular sleeve is based at least in part on a diameter of the first appendage and a desired level of compression enacted by the elastic material of the tubular sleeve on the first appendage. In various embodiments, the tubular sleeve includes a compression strap configured to increase a level of compression enacted by elastic material of the tubular sleeve on the first appendage.

In accordance with another aspect, a protection apparatus for use with a continuous infusion fluid source and a peripheral fluid line is provided. The peripheral fluid line includes an internal portion positioned within a vein located in a first appendage of a patient. The peripheral fluid line further includes an external lumen portion positioned on the exterior of the first appendage and configured to interface with a continuous infusion fluid source. The protection apparatus includes a tubular sleeve of a first length configured to concentrically enclose the first appendage and the external lumen portion of the peripheral fluid line. The tubular sleeve includes a lumen window configured to enable physical and/or visual access to the external lumen portion of the peripheral fluid line.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale, or in schematic in the interest of clarity and conciseness. Not every component may be labeled in every drawing. Any like reference numerals in the figures may represent and refer to the same or similar element or function. In the drawings:

FIG. 1 provides a diagram of an example peripheral fluid line with which an example protection apparatus may be configured for use, in accordance with various embodiments of the present disclosure.

FIG. 2 provides a diagram of an example continuous infusion fluid source with which an example protection apparatus may be configured for use, in accordance with various embodiments of the present disclosure.

FIG. 3 provides an anterior view of an example protection apparatus, in accordance with various embodiments of the present disclosure.

FIG. 4 provides a posterior view of an example protection apparatus, in accordance with various embodiments of the present disclosure.

FIG. 5 provides a coronal/frontal plane view of an example protection apparatus, in accordance with various embodiments of the present disclosure.

FIG. 6 provides another anterior view of an example protection apparatus, in accordance with various embodiments of the present disclosure.

FIG. 7 provides another anterior view of an example protection apparatus, in accordance with various embodiments of the present disclosure.

FIG. 8 provides another coronal/frontal plane view of a pouch portion of an example protection apparatus, in accordance with various embodiments of the present disclosure.

FIG. 9 provides another anterior view of an example protection apparatus, in accordance with various embodiments of the present disclosure.

### DETAILED DESCRIPTION OF VARIOUS EMBODIMENTS

Described herein are various embodiments of a protection apparatus configured for use with a peripheral fluid line, such as a PICC, and a continuous infusion fluid source, such as a balloon pump. In various embodiments, the protection apparatus comprises a tubular sleeve configured to be worn on an appendage of an adult person within which the peripheral fluid line is positioned. In various embodiments, the peripheral fluid line may comprise an external lumen portion protruding from an appendage of an adult person, and the tubular sleeve is configured to be worn over the external lumen portion, such that the external lumen portion is unexposed and protected. In various embodiments, the tubular sleeve comprises a pouch portion configured to secure a continuous infusion fluid source, such as a balloon pump. Thus, as described in greater detail herein, the protection apparatus comprises a tubular sleeve with a pouch portion configured to protect and surround at least one continuous infusion fluid source, thereby providing various technical advantages including the reduction of pain related to the peripheral fluid line experienced by the adult person and the increased mobility of the adult person while maintaining the continuous infusion of medication through the peripheral fluid line.

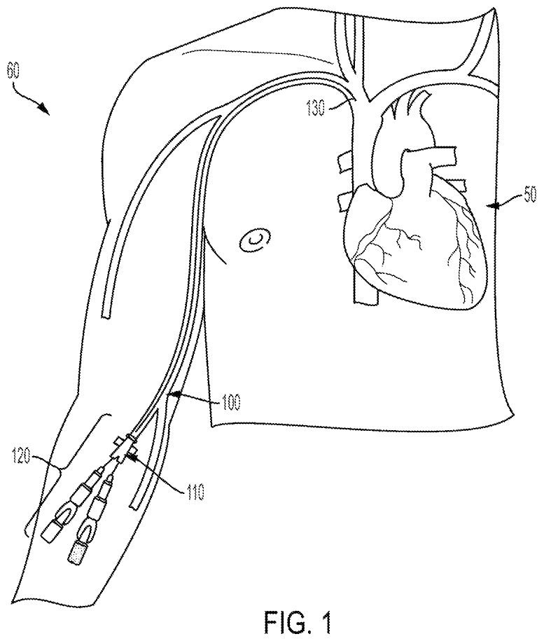

In various embodiments, the protection apparatus is configured for use with a peripheral fluid line positioned within a peripheral or appendage of an adult person. FIG. 1 illustrates a diagram of an example peripheral fluid line 100 with which the protection apparatus is configured for use. As illustrated, the peripheral fluid line 100 may be an intravenous (IV) line, a central IV line, a catheter, a central catheter, a PICC, and/or the like positioned within a vein of an individual or patient. Specifically, at least a portion of the peripheral fluid line 100 may be positioned within a peripheral vein or a vein of the peripheral vascular system (e.g., a portion of the circulatory system comprising veins and arteries not in the chest or abdomen of an individual or patient). The vein within which the peripheral fluid line 100 is positioned may span through an appendage 60 of the patient to the heart 50 of the patient, and thus, the peripheral fluid line 100 may be configured to administer fluids (e.g., fluid medication) to the heart 50 of the patient. In various embodiments, a substantial portion of the peripheral fluid line 100 including a first or distal end 110 of the peripheral fluid line 100 may be located at the appendage 60. In various embodiments, such as the illustrated embodiment, a second or proximal end 130 of the peripheral fluid line 100 may be positioned within a chest or abdominal region substantially near the heart of the patient (e.g., the peripheral fluid line 100 is a central catheter). In other embodiments, a second or proximal end 130 of the peripheral fluid line 100 may be located at the appendage 60, and thus, the peripheral fluid line 100 may only span the appendage 60. It will be understood that while in the illustrated embodiment, the appendage 60 is an arm of the individual or patient, a peripheral fluid line 100 may be configured for use with and positioned within other appendages 60, such as a leg.

At the distal end 110, the peripheral fluid line 100 may comprise an external lumen portion 120. In various embodiments, the external lumen portion 120 is positioned external to the appendage 60. For example, the external lumen portion 120 may protrude from the appendage 60 through an incision made on the appendage 60. In some instances, a port may be installed on or attached to the skin of the appendage 60, and the external lumen portion 120 may protrude through the port.

In various embodiments, the external lumen portion 120 is configured to interface with a continuous infusion fluid source, such as a balloon pump, such that fluids (e.g., fluid medication) stored within the medication source may travel into and through the peripheral fluid line 100. Accordingly, the external lumen portion 120 may comprise various lumen, seals, valves, claves, septum, housings, caps, screws, needles, and/or the like configured to prevent leakage at the interface between the continuous infusion fluid source and the external lumen portion 120 and to prevent external contaminants from entering the peripheral fluid line 100. In various embodiments, each lumen of the external lumen portion 120 may be configured to interface with a continuous infusion fluid source, or balloon pump, and the external lumen portion 120 may comprise one or more lumen. In the illustrated embodiment, for example, the external lumen portion 120 comprises two lumen, and as such, two continuous infusion fluid sources may interface with the peripheral fluid line 100 and provide fluids into and through the peripheral fluid line 100.

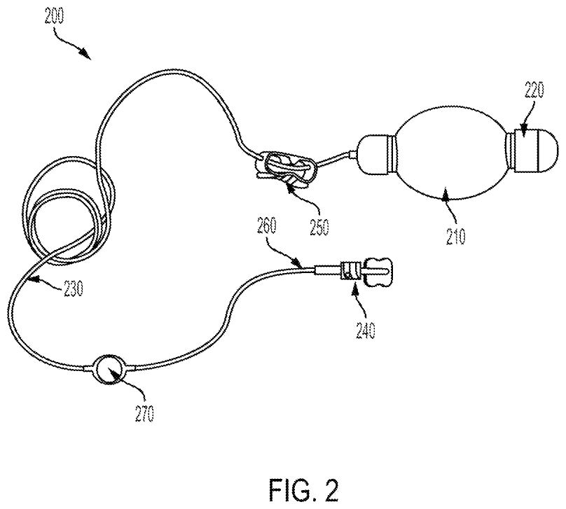

In various embodiments, the continuous infusion fluid source is a balloon pump storing fluid medication, and the peripheral fluid line 100 is configured to interface with one or more balloon pumps based at least in part on the number of lumens at the external lumen portion 120. It will be appreciated that, in various embodiments, the protection apparatus may be configured for use with various other continuous infusion fluid sources, such as intravenous bags, reservoirs, drip bags, pumps, syringes, and/or the like. In any regard, the following figures may illustrate an example continuous infusion fluid source being a balloon pump, though these illustrations and associated descriptions should not be construed as limiting as to what continuous infusion fluid sources may be used with various embodiments described herein. Thus, FIG. 2 illustrates an example continuous infusion fluid source being a balloon pump 200.

In various embodiments, the balloon pump 200 comprises a fluid storage balloon 210. The fluid storage balloon 210 may comprise one or more layers, and the innermost layer within which fluid medication may be contained may comprise elastic and/or elastomeric materials. In various embodiments, the balloon pump 200 further comprises a fill port 220. The fill port 220 may be configured to enable fluids (e.g., fluid medication) to be inserted (e.g., pumped) into the innermost layer of the fluid storage balloon 210. Accordingly, the innermost layer is caused to generate a compressive pressure on the contained fluid, as volumes of fluid above a specific volume cause the elastic and/or elastomeric material of the innermost layer to stretch. Thus, the fluid storage balloon 210 is configured to compress the contained fluid such that the contained fluid is ejected through a pump line 230. In various embodiments, the pressure causing the fluid (e.g., fluid medication) to be ejected through the pump line 230 may be variable over time. For instance, lower volumes of fluid within the fluid storage balloon 210 after ejection of fluid may not stretch the innermost layer to the previous extent, and thereby may not generate as much compressive pressure.

Fluid may be ejected from the fluid storage balloon 210 through the pump line 230 and out through a pump line end 240. In various embodiments, the pump line end 240 is configured to interface with a lumen of the external lumen portion 120 of a peripheral fluid line 100. As such, fluid (e.g., fluid medication) travels through the pump line end 240 and through a lumen into a peripheral fluid line 100.

Thus, fluids may be administered to a patient using a continuous infusion fluid source, such as the illustrated balloon pump 200, through a peripheral fluid line 100. It will be appreciated that another continuous infusion fluid source, which may include a different fluid reservoir and pumping mechanism than the fluid storage balloon 210 but may still include a pump line 230 and means (e.g., a pump line end 240) to interface with the peripheral fluid line 100. In various embodiments, the continuous infusion fluid source (e.g., balloon pump 200) comprises additional components that may regulate and/or enable the control of fluid flow through the pump line 230. For example, balloon pump 200 comprises a clamp 250, a flow restrictor 260, and a filter 270.

Exemplary Protection Apparatus

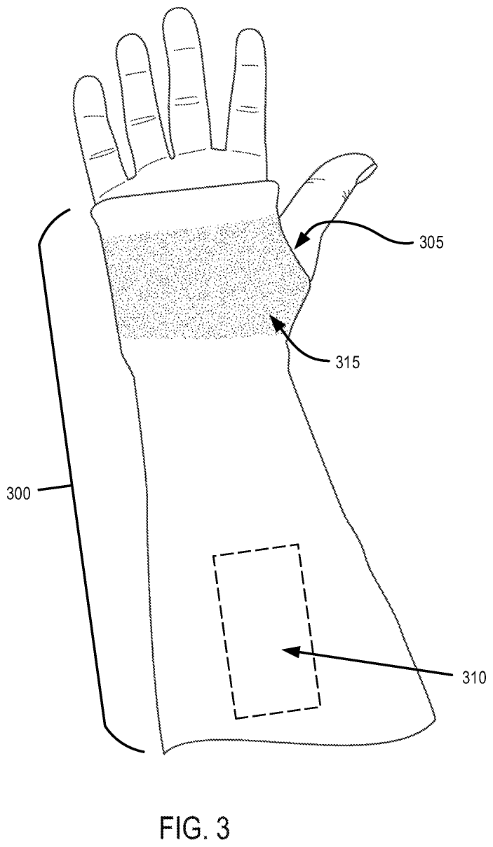

Having thus described a peripheral fluid line 100 and a continuous infusion fluid source, such as a balloon pump 200, with which a protection apparatus may be used, FIGS. 3-9 provide various views or perspectives of example protection apparatuses and portions thereof. In various embodiments, a protection apparatus is configured to secure a continuous infusion fluid source while maintaining a fluid connection between the continuous infusion fluid source and the peripheral fluid line. In various embodiments, a protection apparatus comprises a tubular sleeve configured to be worn on an appendage 60 of a patient. FIG. 3 illustrates an anterior view of a tubular sleeve 300. That is, FIG. 3 illustrates an anterior-facing aspect of the tubular sleeve 300.

In the illustrated embodiment, the tubular sleeve 300 is worn on the right arm of the patient, but it will be understood that the tubular sleeve 300 may be worn on any appendage 60 of the patient. Specifically, the tubular sleeve 300 is worn on the appendage 60 at which the external lumen portion 120 of a peripheral fluid line 100 is positioned. Thus, in the illustrated embodiment, the external lumen portion 120 of a peripheral fluid line 100 is positioned on the right arm of the patient, although not explicitly shown. The tubular sleeve 300 may be composed of any material suitable for being worn on a patient. For example, the tubular sleeve 300 may be composed of fabric material. In various embodiment, the tubular sleeve 300 is composed of elastic fabric material, thereby increasing the ease of putting on the tubular sleeve 300 while ensuring the tubular sleeve 300 is secured and substantially immobilized once worn on the appendage 60.

1. Tubular Sleeve Dimensions

In various embodiments, the tubular sleeve 300 is configured with a radius or diameter based at least in part on the radius or diameter of the appendage 60. For example, a tubular sleeve 300 for wear on a leg may have a greater radius or diameter than a tubular sleeve 300 for wear on an arm. The radius or diameter of the tubular sleeve 300 may be greater than the radius or diameter of the appendage 60 such that the tubular sleeve 300 may be pulled onto the appendage 60 (e.g., concentrically).

In some embodiments, an average circumference of the tubular sleeve 300 may be greater than or equal to approximately 5 inches. In some embodiments, the average circumference of the tubular sleeve 300 is less than or equal to approximately 15 inches. In such embodiments, the tubular sleeve 300 may be configured for wear on an arm or forearm of a patient. In some embodiments, the circumference of the tubular sleeve 300 may vary similar to the morphology of an arm or forearm. For example, a circumference of the distal end of a tubular sleeve 300 may be less than the circumference of the proximal end of the tubular sleeve 300.

As shown in the illustrated embodiment, the tubular sleeve 300 extends longitudinally along the right arm. In various embodiments, a proximal end of the tubular sleeve 300 extends to a configurable position on the appendage 60 (e.g., the right arm). For example, the proximal end of the tubular sleeve 300 may be positioned at the elbow region of the right arm, so the tubular sleeve 300 extends longitudinally in a proximal direction to the elbow region of the right arm. In another example, the proximal end of the tubular sleeve 300 may be positioned at a shoulder region of the right arm, so the tubular sleeve 300 extends longitudinally in a proximal direction to the shoulder region of the right arm. As a further example, the tubular sleeve 300 is worn on a leg and extends longitudinally along the leg in a proximal direction to a hip or groin region (e.g., the proximal end of the tubular sleeve 300 is positioned at the hip or groin region). As an even further example, the tubular sleeve is worn on a leg and extends longitudinally along the leg in a proximal direction to a knee region (e.g., the proximal end of the tubular sleeve 300 is positioned at the knee region).

In various embodiments, the tubular sleeve 300 may comprise one or more longitudinal portions which may be configured to be attachable (and detachable). For example, a first longitudinal portion of the tubular sleeve 300 may extend to an elbow region of the appendage, and a second longitudinal portion of the tubular sleeve 300 may extend from the elbow region to a shoulder region of the appendage. In such an example, the second longitudinal portion may be configured to attach via attachment means to the first longitudinal portion, and likewise may be configured to detach via the same means and/or detachment means from the first longitudinal portion. Such attachment and/or detachment means configured to attach and/or detach longitudinal portions of the tubular sleeve with each other may include zippers, latches, buckles, hook-and-loop fasteners (e.g., Velcro), fasteners, pins, clips, belts, ribbons, strings, adhesives, and/or the like.

As such, the length of the tubular sleeve 300 may be configurable. In some embodiments, a first length of the tubular sleeve 300 may be at least approximately 10 inches. In some embodiments, the first length of the tubular sleeve 300 may be less than or equal to approximately 18 inches. In various embodiments, the tubular sleeve 300 may be extended to a second length (e.g., via an attachable longitudinal portion) less than or equal to approximately 32 inches.

In various embodiments, one or more longitudinal portions of the tubular sleeve 300 may be configured to attach to (and detach from) an article of clothing, garment, apparel, and/or the like worn by the patient. As such, a longitudinal portion of the tubular sleeve 300 and/or the tubular sleeve 300 itself may comprise attachment and/or detachment means (such as the above described attachment means) for attaching to and/or detaching from an article of clothing, garment, apparel, and/or the like worn by the patient. For example, a tubular sleeve 300 worn on an arm of the patient may be configured to attach via attachment means to a shirt worn by the patient and/or a shirt sleeve of the shirt worn by the patient (e.g., at a shoulder region, at an elbow region). As another example, a tubular sleeve 300 worn on the leg of the patient may be configured to attach via attachment means to a pair of pants worn by the patient and/or a pant leg of the pair of pants worn by the patient. It may be appreciated that in general, such attachment and/or detachment means may be located at the proximal end of the tubular sleeve 300, as the tubular sleeve 300 is worn on an appendage 60.

In various embodiments, the tubular sleeve 300 may extend longitudinally in a distal direction such that the tubular sleeve 300 is a configurable length (e.g., measured from the distal end of the tubular sleeve 300 to the proximal end of the tubular sleeve 300). In various embodiments, the distal end of the tubular sleeve 300 may extend to a distal end of the appendage 60. For example, in the illustrated embodiment, the tubular sleeve 300 extends to the right hand of the right arm. As another example, a tubular sleeve 300 worn on a leg may extend to an ankle or a foot.

In various embodiments, the tubular sleeve 300 comprises appendage attachments at least at the distal end of the tubular sleeve 300. For example, the tubular sleeve 300 comprises a thumb hole 305 and/or finger holes, thereby securing the distal end of the tubular sleeve 300 to the appendage 60. The thumb and/or fingers may protrude through the thumb hole 305 and/or finger holes. Such thumb holes 305 and/or finger holes provide further advantages by enabling the patient to be uninhibited from performing various simple tasks (e.g., operating a smartphone) while wearing the tubular sleeve 300. As another example, a tubular sleeve 300 worn on a leg may comprise an elastic band to secure the distal end of the tubular sleeve 300 to the appendage 60.

2. Lumen Window

In various embodiments, the tubular sleeve 300 is configured to be worn over (e.g., circumferentially enclose) the external lumen portion 120, such that the external lumen portion 120 is positioned underneath the tubular sleeve 300, radially internal to the tubular sleeve 300, between the tubular sleeve 300 and the appendage 60, and/or other equivalent positioning references. The tubular sleeve 300 may comprise a lumen window 310 substantially near and/or co-located with the external lumen portion 120 when being worn on the appendage 60. In the illustrated embodiment, the external lumen portion 120 may be positioned on the inward-facing aspect of the forearm, and the lumen window 310 is accordingly positioned on a portion of the tubular sleeve 300 co-located with the inward-facing aspect of the forearm. Thus, the lumen window 310 may be positioned above or radially external to the external lumen portion 120.

In various embodiments, the lumen window 310 enables access to the external lumen portion 120 of the peripheral fluid line while the tubular sleeve 300 is being worn. Accordingly, the lumen window 310 provides technical advantages, such as in scenarios when the external lumen portion 120 needs to be adjusted, cleaned, or examined and/or when a fluid connection via the external lumen portion 120 between the continuous infusion fluid source and the peripheral fluid line needs to be examined or tested. Accordingly, the lumen window 310 may be configured to enable access to the external lumen portion 120. In some embodiments, the lumen window 310 may be a portion of the tubular sleeve 300 composed of transparent material (e.g., plastic) such that the external lumen portion 120 may be visually examined.

Additionally or alternatively, the lumen window 310 may be a portion of the tubular sleeve 300 configured to be removed and/or displaced for physical access to the external lumen portion 120. For example, the lumen window 310 may be a flap, where opening of such flap allows physical (and visual) access to the external lumen portion 120. Various embodiments may include any configuration or variation of such flap, wherein a side of a rectangular flap may be opened (e.g., three sides of the flap remain attached or connected to the tubular sleeve 300), a corner of the rectangular flap may be opened (e.g., two sides of the flap remain attached or connected to the tubular sleeve 300), and/or the entire rectangular flap may be opened (e.g., one side of the flap remains attached or connected to the tubular sleeve 300). Other configurations of the lumen window 310 may include a slot or slit through which physical (and visual) access to the external lumen portion 120 is granted.

In any configuration of the lumen window 310, various attachment means (such as the attachment means defined previously) may be configured to otherwise prevent access to the external lumen portion 120 barring interaction. For example, an interactable attachment mean may be a Velcro strip securing a flap in a state obscuring the external lumen portion 120 until interaction with the Velcro strip and/or the flap grants access to the external lumen portion 120. Thus, the lumen window 310 may be configured in any manner to selectively and situationally provide access to the external lumen portion 120. The lumen window 310 may also be configured in any shape (e.g., elliptical, rectangular) and may be any size large enough for access to the external lumen portion 120.

3. Pouch Portion

The tubular sleeve 300 may also comprise a pouch portion 315 configured to define a volume within which a continuous infusion fluid source may be positioned and secured. In various embodiments, the pouch portion 315 is located substantially near and/or at the distal end of the tubular sleeve 300. For example, in the illustrated embodiments, the pouch portion 315 is located near the distal end of the tubular sleeve 300 such that the pouch portion 315 is co-located with the palmar aspect of the right hand of the right arm of the patient. The pouch portion 315 may be located at a configurable distance from the lumen window 310 based at least in part on the continuous infusion fluid source. Specifically, the pouch portion 315 is located at a distance from the lumen window 310 such that a continuous infusion fluid source positioned within a volume defined by the pouch portion 315 may reach and maintain a fluid connection with the external lumen portion 120 co-located with the lumen window 310. For example, the pump line 230 of the balloon pump 200 may be of a specific and/or standardized length, and the pouch portion 315 may be located at a distance from the lumen window 310 based at least in part on the length of the pump line 230.

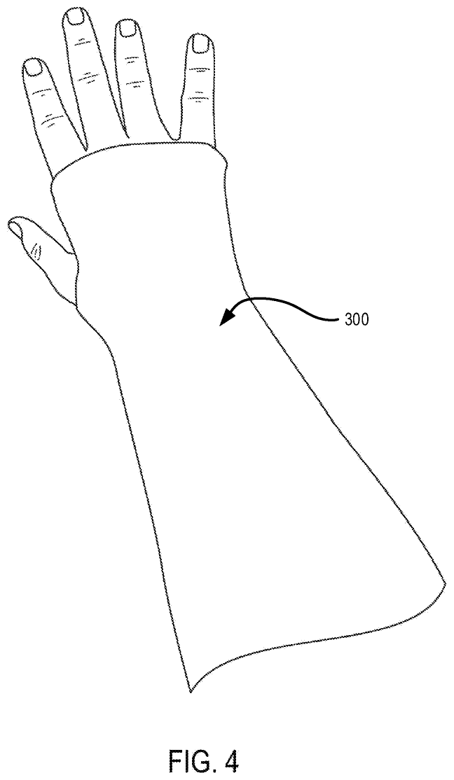

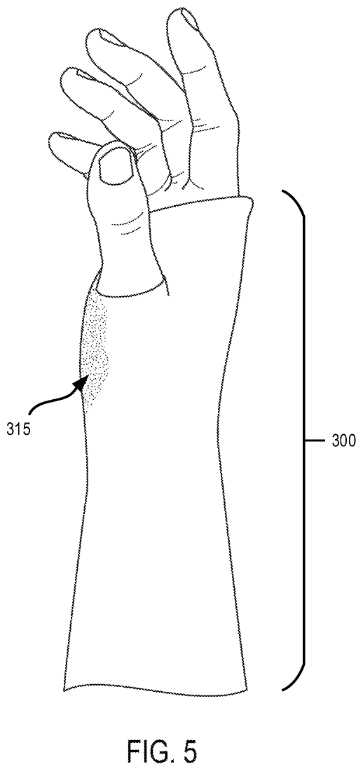

While the pouch portion 315 is located at and/or near a distal end of the tubular sleeve 300, the pouch portion 315 may be configured with a circumferential length, in various embodiments. For example, FIG. 4 provides a posterior view of the tubular sleeve 300, or a view of the posterior-facing aspect of the tubular sleeve 300. The pouch portion 315 may extend circumferentially to span approximately half of the circumference of the tubular sleeve 300, such that the pouch portion 315 is located on the anterior-facing aspect of the tubular sleeve 300. Thus, a substantial part of pouch portion 315 may not be located on the posterior-facing aspect of the tubular sleeve 300 illustrated in FIG. 4. FIG. 5 provides a coronal/frontal plane view of the tubular sleeve 300, which further illustrates the pouch portion 315 being largely located on the anterior-facing aspect of the tubular sleeve 300.

It may be appreciated that the pouch portion 315 being located on the anterior-facing aspect of the tubular sleeve 300 may be advantageous at least when the external lumen portion 120 is positioned on an anterior-facing aspect of the appendage 60 (e.g., the right arm). In such instances, the pouch portion 315 and the lumen window 310 may be axially aligned along the longitudinal axis of the appendage 60 and the tubular sleeve 300, and by extension, a continuous infusion fluid source positioned and secured within the volume defined by the pouch portion 315 and the external lumen portion 120 may also be axially aligned.

In some embodiments however, the pouch portion 315 is located on the posterior-facing aspect of the tubular sleeve 300 and may be co-located with the dorsal aspect of the hand, although not explicitly illustrated in FIG. 4. In such embodiments, the pouch portion 315 may also span approximately half of the circumference of the tubular sleeve 300 while being co-located with the dorsal aspect of the hand.

In various other embodiments, the pouch portion 315 may circumferentially span the entire circumference of the tubular sleeve 300 at and/or near a longitudinal distal end of the tubular sleeve 300. For example, the pouch portion 315 may be co-located with both the palmar aspect and the dorsal aspect of the hand. In various other embodiments, the tubular sleeve comprises one or more pouch portions 315, where one pouch portion 315 may be positioned at the palmar region and span less than half of the circumference of the tubular sleeve and another pouch portion 315 may be positioned at the dorsal aspect of the hand and span less than half of the circumference of the tubular sleeve. Those of ordinary skill in the art may imagine any configuration of pouch portion(s) 310 including circumferential spans of each pouch portion 315.

In various embodiments, the tubular sleeve 300 is ambidextrous, which may allow for further configurations and positioning of one or more pouch portions 315. Returning to the embodiment in which a pouch portion 315 is co-located with a palmar aspect of a hand of a first arm and spans approximately half of the circumference of the tubular sleeve 300, the tubular sleeve 300 may be worn on the other arm of the patient such that the pouch portion 315 is positioned at the dorsal aspect of the hand of the other arm. For example, FIG. 3 illustrates the tubular sleeve being worn on the right arm of the patient with the pouch portion 315 being co-located with the palmar aspect of the right hand; however, the illustrated tubular sleeve may also be configured to be worn on the left arm of the patient, resulting in the pouch portion 315 being co-located with the dorsal aspect of the left hand.

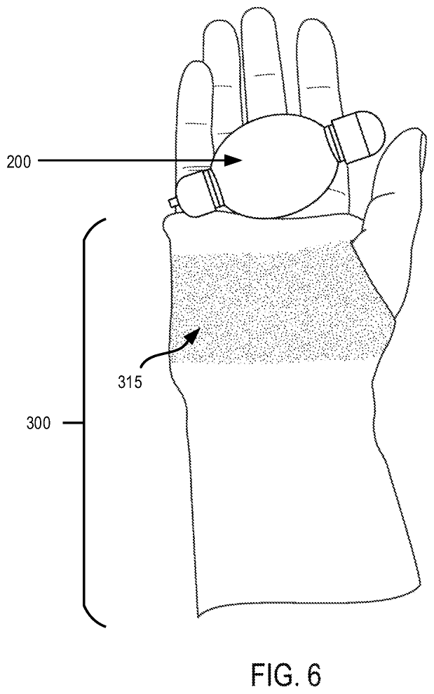

In various embodiments, the pouch portion 315 is dimensioned (e.g., circumferential span, longitudinal length, thickness) based at least in part on the continuous infusion fluid source, such as the balloon pump 200. For example, the pouch portion 315 may be dimensioned such that the balloon pump 200 may be positioned within a volume defined by the pouch portion 315. At least the circumferential span of the pouch portion 315 across the tubular sleeve and the longitudinal length of the pouch portion 315 along the tubular sleeve may be configured such that the pouch portion 315 defines a volume greater than or equal to the continuous infusion fluid source (e.g., the balloon pump 200). FIG. 6 illustrates the balloon pump 200 and the pouch portion 315, where the pouch portion 315 is dimensioned such that the balloon pump 200 may be positioned within the volume defined by the pouch portion 315.

The volume of the pouch portion 315 may be defined between the pouch portion and the co-located anatomical portion of the patient. Returning to the illustrated embodiment of FIG. 3, the pouch portion 315 may be a portion of the tubular sleeve, and a volume is defined between the palmar aspect of the patient's hand and the pouch portion 315. Accordingly, the pouch portion 315 may be dimensioned that the balloon pump 200 may be positioned between the palmar aspect of the patient's hand and the pouch portion 315.

In various other embodiments, the pouch portion 315 may be attached to the tubular sleeve and define a volume between the pouch portion 315 and the tubular sleeve. For example, the pouch portion 315 may be a planar material attached (e.g., sewn) to the exterior of the tubular sleeve to define a volume within the attachments and between the tubular sleeve and the attached material. As another example, the pouch portion 315 may be attached to the interior of the tubular sleeve to define a volume within the attachments and between the tubular sleeve and the attached material. In some embodiments, the tubular sleeve comprises two pouch portions 315 co-located with the palmar aspect of the hand, with one being attached to the exterior of the tubular sleeve and another being attached to the interior of the tubular sleeve.

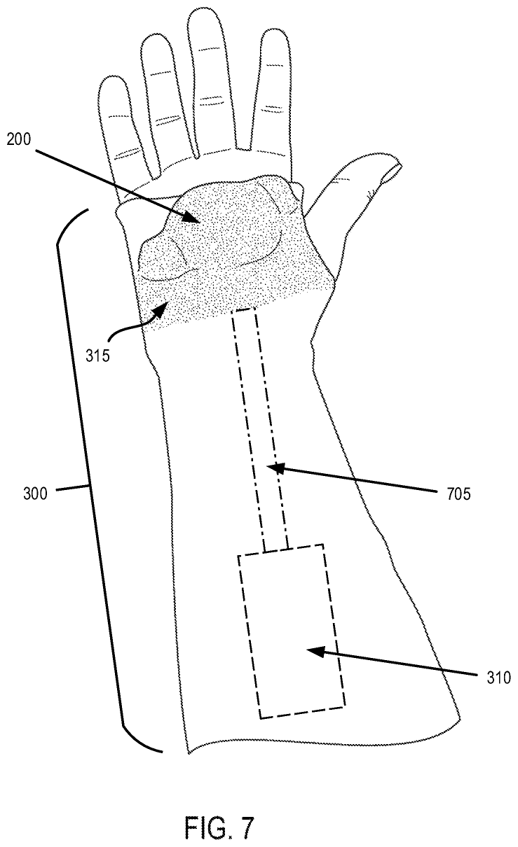

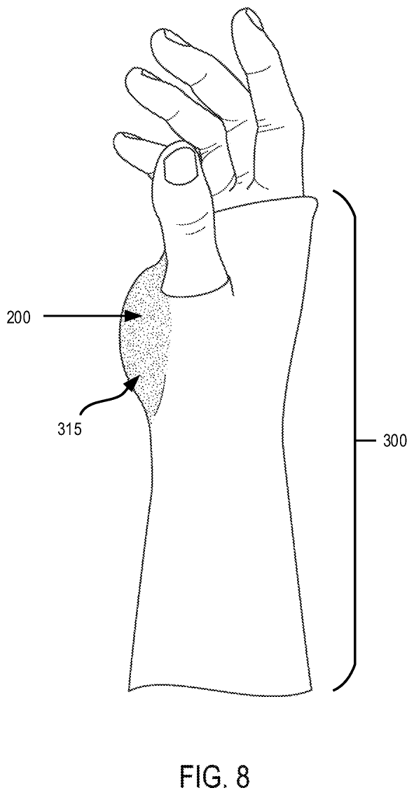

Accordingly, FIGS. 7 and 8 illustrate the balloon pump 200 being positioned within a volume defined by the pouch portion 315. In various embodiments, the pouch portion 315 is configured to enable the balloon pump 200 to be inserted into the volume defined by the pouch portion 315. For example, the pouch portion 315 may be composed of elastic material, such that the balloon pump 200 may be inserted into the volume defined by the pouch portion 315 by elastically expanding the pouch portion 315. A patient wearing the tubular sleeve may manually stretch the elastic material of the pouch portion 315, thereby defining or expanding an entry into the volume defined by the pouch portion 315, and subsequently insert the balloon pump 200 into the defined volume via the entry. In various embodiments, the pouch portion 315 is configured with insertion means enabling the balloon pump 200 to be inserted into the volume defined by the pouch portion 315. For example, the pouch portion 315 may comprise a zipper, a latch, a flap, a fold, a button, and/or the like, such that an entry into the volume may be dynamically created and through which the balloon pump 200 may be inserted.

Furthermore, the pouch portion 315 may be configured to secure the balloon pump 200 within the volume defined by the pouch portion 315. Such a configuration of the pouch portion 315 provides various advantages by reducing unintended or spontaneous movements of the balloon pump 200 during infusion and by preventing the balloon pump 200 from being inadvertently ejected, removed, or dis-positioned from the volume defined by the pouch portion 315. For example, the pouch portion 315 may comprise a retention mechanism such as an internal ring within the defined volume configured to immobilize the balloon pump 200. As another example, the pouch portion 315 may comprise an enlarged seam sewn into the elastic and/or fabric material of the pouch portion 315 such that the enlarged seam prevents movement of the balloon pump 200 within and/or out of the defined volume. As a further example, an interior facing aspect of the pouch portion 315 (e.g., internal to the defined volume) may be composed of a rubber or high-friction material that substantially immobilizes the balloon pump 200 when in physical contact with the balloon pump 200.

While the continuous infusion fluid source (e.g., a balloon pump 200) is positioned and secured within the pouch portion 315, the tubular sleeve 300 may be configured to maintain and secure the fluid connection between the continuous infusion fluid source and the external lumen portion 120 of the peripheral fluid line 100. As aforementioned, the balloon pump 200 and/or another continuous infusion fluid source may comprise a pump line 230 that may attach at a pump line end 240 to the external lumen portion 120 to provide fluids to the peripheral fluid line 100. Accordingly, the tubular sleeve 300 may be configured to secure (e.g., immobilize) a pump line 230 or other fluid line between the continuous infusion fluid source and the external lumen portion 120. In various embodiments, the tubular sleeve 300 is composed of elastic material and uses elastic force to secure and immobilize the pump line 230. In such embodiments, the pump line 230 may be secured from elastic force against the appendage 60 by the tubular sleeve 300.

In various embodiments, the tubular sleeve 300 may comprise a longitudinal channel 705 spanning from the pouch portion 315 to the lumen window 310 within which the pump line 230 may be positioned. As such, a width or a diameter of the longitudinal channel 705 may be configured to enable a pump line 230 to be positioned within the longitudinal channel 705. As aforementioned, the pouch portion 315 and the lumen window 310 may be axially aligned, thereby resulting in the longitudinal channel 705 being parallel to a longitudinal axis of the appendage 60 and/or the tubular sleeve 300. The longitudinal channel 705 may be positioned internal to the tubular sleeve 300 or external to the tubular sleeve 300. In some embodiments, the longitudinal channel 705 comprises one or more retention clips configured to secure the pump line 230 within the longitudinal channel 705.

Exemplary Advantages

Having thus described various embodiments of a protection apparatus comprising a tubular sleeve 300 configured to secure a continuous infusion fluid source (e.g., a balloon pump 200) and maintain a fluid connection (e.g., via pump line 230) between the continuous infusion fluid source and an external lumen portion 120 of a peripheral fluid line during infusion, various technical advantages of such a protection apparatus may be apparent to those of skill in the art to which the present disclosure pertains. The tubular sleeve 300 may specifical reduce pain experienced by the patient during infusion by securing and immobilizing each of the balloon pump 200, the pump line 230, and the external lumen portion 120, for example. Such objects are further secured against the appendage 60 of the patient, such that the tubular sleeve 300 enables mobility of the patient.

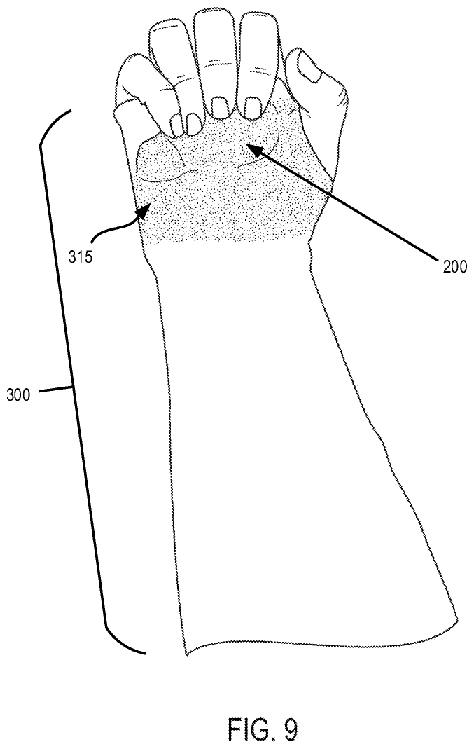

Furthermore, the tubular sleeve 300 advantageously enables the relatively normal use of the appendage 60 during infusion. In FIG. 9 for example, the patient may flex the fingers of the hand during infusion with the balloon pump 200 positioned, secured, and immobilized within the volume defined by the pouch portion 315, and thus, the patient may perform various tasks that require the use of fingers. As such, the tubular sleeve 300 and the pouch portion 315 may unencumber the fingers of the patient, which in existing systems, may be grasping the balloon pump 200. For example, the patient may use the fingers unencumbered by the tubular sleeve 300 and the pouch portion 315 to operate a smartphone, pull open a door, and/or various other tasks.

The tubular sleeve 300 and the pouch portion 315 may further enable the patient to manipulate the flow of the fluids from the balloon pump 200. As also shown in FIG. 9, the patient may use fingers and/or the hand to physically compress the balloon pump 200 to cause fluid to be ejected from the fluid storage balloon 210. Enablement of the patient to manipulate the flow of the fluids (e.g., increasing the flow) may be particular advantageous in instances when a fluid level within the fluid storage balloon 210 is low and/or depleted, and additional compression force in addition to the elastic compression of the fluid storage balloon 210 is needed to provide a required amount of fluids to the peripheral fluid line 100.

Exemplary Processes

Thus, the tubular sleeve 300 may provide various technical advantages and may specifically exhibit such technical advantages when worn by the patient on an appendage 60 on which the external lumen portion 120 of a peripheral fluid line 100 is positioned. The patient may perform various steps/operations to wear the tubular sleeve 300 and configure the tubular sleeve 300 to secure and immobilize at least a continuous infusion fluid source.

In a first example process, the patient may enclose the appendage 60 with the tubular sleeve 300. In various embodiments, the tubular sleeve 300 may be pulled onto the appendage 60 (e.g., concentrically) from a distal end of the appendage 60 to thereby circumferentially enclose the appendage 60. The patient may pull the tubular sleeve 300 onto the appendage 60 to a certain position along the appendage 60. For example, the patient may pull the tubular sleeve 300 onto the appendage 60 such that the lumen window 310 is aligned with and/or co-located with the external lumen portion 120.

In various embodiments, the tubular sleeve 300 may be comprise a longitudinal opening to assist the patient in enclosing the appendage 60 with the tubular sleeve 300. In some embodiments, the longitudinal opening may span the longitudinal length of the tubular sleeve 300, and thus, the tubular sleeve 300 may be expanded and opened into a rectangular planar object. The patient may then wrap the opened tubular sleeve 300 around the appendage 60 and interact with various mechanisms to cause the tubular sleeve 300 to return and stay in a tubular configuration. For example, the longitudinal opening may be closed via a zipper. In some other embodiments, the longitudinal opening may only span a portion of the longitudinal length of the tubular sleeve 300.

In various embodiments, the tubular sleeve 300 may be secured and/or immobilized at a position along the appendage 60. For example, the tubular sleeve 300 may comprise a compression strap that may be tightened such that the tubular sleeve 300 is longitudinally immobilized along the appendage 60. Additionally or alternatively, the tubular sleeve 300 may be composed of elastic material of sufficient elasticity to conform to the appendage 60 and remain longitudinally immobilized along the appendage 60.

Once the tubular sleeve 300 is enclosing the appendage 60, the patient may insert the continuous infusion fluid source (e.g., balloon pump 200) into the volume defined by the pouch portion 315. As previously described, the pouch portion 315 may comprise various means to generate and/or expand an entry via which the continuous infusion fluid source may be inserted into the defined volume.

Subsequently, the patient may connect a pump line 230 of the continuous infusion fluid source to the external lumen portion 120 via the lumen window 310. As aforementioned, the lumen window 310 may enable physical access to the external lumen portion 120, and the patient may thus connect the pump line 230 to the external lumen portion 120 via the lumen window 310. In some embodiments, the pump line 230 may be first fed through a longitudinal channel 705. The patient may then begin infusion after the pump line 230 of the continuous infusion fluid source is connected to the external lumen portion 120 and a fluid connection is formed.

In another example process, the patient may first form a fluid connection between the continuous infusion fluid source and the external lumen portion 120 of the peripheral fluid line and subsequently enclose the appendage 60 in the tubular sleeve 300. In some embodiments, the patient may ensure that the tubular sleeve 300 encloses the appendage 60 such that the lumen window 310 is aligned with and/or co-located with the external lumen portion 120. In some embodiments, the patient may ensure that the continuous infusion fluid source and the external lumen portion 120 remain axially aligned after being enclosed by the tubular sleeve 300.

### CONCLUSION

Many modifications and other embodiments of the present disclosure set forth herein will come to mind to one skilled in the art to which the present disclosures pertain having the benefit of the teachings presented in the foregoing descriptions and the associated drawings. Therefore, it is to be understood that the present disclosure is not to be limited to the specific embodiments disclosed and that modifications and other embodiments are intended to be included within the scope of the appended claim concepts. Although specific terms are employed herein, they are used in a generic and descriptive sense only and not for purposes of limitation.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above. Accordingly, the specification is intended to embrace all such modifications and variations of the disclosed embodiments that fall within the spirit and scope of the appended claims.

In addition, various inventive concepts may be embodied as one or more methods, of which an example has been provided. The acts performed as part of the method may be ordered in any suitable way. Accordingly, embodiments may be constructed in which acts are performed in an order different from illustrated, which may include performing some acts simultaneously, even though shown as sequential acts in illustrative embodiments.

That which is claimed:

1. A protection apparatus for use with a continuous infusion fluid source and a peripheral fluid line, wherein the peripheral fluid line comprises an internal portion positioned within a vein located in a first appendage of a patient and an external lumen portion positioned on the exterior of the first appendage and configured to interface with a continuous infusion fluid source, the protection apparatus comprising:
a tubular sleeve of a first length configured to concentrically enclose the first appendage and the external lumen portion of the peripheral fluid line,
wherein the tubular sleeve comprises a pouch portion located at a distal end of the tubular sleeve, the pouch portion defining a volume configured to contain and secure the continuous infusion fluid source.

2. The protection apparatus of claim 1, wherein the tubular sleeve comprises a lumen window configured to enable physical and/or visual access to the external lumen portion of the peripheral fluid line.

3. The protection apparatus of claim 2, wherein the lumen window comprises a flap configured to be opened to enable physical access to the external lumen portion of the peripheral fluid line.

4. The protection apparatus of claim 2, wherein the tubular sleeve comprises a longitudinal channel spanning a distance from the pouch portion to the lumen window and configured to secure a fluid connection line connecting the continuous infusion fluid source and the external lumen portion of the peripheral fluid line.

5. The protection apparatus of claim 1, wherein the tubular sleeve is configured to attach with and/or detach from an article of clothing worn by the patient.

6. The protection apparatus of claim 1, wherein the tubular sleeve is configured to attach with and/or detach from an additional tubular longitudinal portion such that the tubular sleeve is a second length different than the first length.

7. The protection apparatus of claim 1, wherein the pouch portion circumferentially spans approximately half of the circumference of the tubular sleeve and is located on an anterior-facing aspect of the tubular sleeve.

8. The protection apparatus of claim 7, wherein the pouch portion and the lumen window are axially aligned along an axis substantially parallel to a longitudinal axis of the tubular sleeve.

9. The protection apparatus of claim 7, wherein the tubular sleeve is ambidextrous, and the pouch portion is co-located with a posterior-facing or dorsal aspect of a second appendage.

10. The protection apparatus of claim 1, wherein the pouch portion is composed of elastic material and is configured to expand to a state in which the continuous infusion fluid source may be inserted into the volume defined by the pouch portion.

11. The protection apparatus of claim 1, wherein the pouch portion comprises an expansion mechanism configured to cause the pouch portion to expand to a state in which the continuous infusion fluid source may be inserted into the volume defined by the pouch portion.

12. The protection apparatus of claim 1, wherein the pouch portion comprises a retention mechanism configured to immobilize the continuous infusion fluid source within the volume defined by the pouch portion.

13. The protection apparatus of claim 1, wherein the appendage is an arm of the patient, and the volume defined by the pouch portion is substantially co-located with a palmar region of the hand of the arm while the tubular sleeve concentrically encloses the arm.

14. The protection apparatus of claim 9, wherein the tubular sleeve comprises one or more openings through which one or more digits of the hand may protrude while the tubular sleeve concentrically encloses the arm.

15. The protection apparatus of claim 1, wherein the tubular sleeve is composed of elastic material.

16. The protection apparatus of claim 13, wherein a diameter of the tubular sleeve is based at least in part on a diameter of the first appendage and a desired level of compression enacted by the elastic material of the tubular sleeve on the first appendage.

17. The protection apparatus of claim 13, wherein the tubular sleeve comprises a compression strap configured to increase a level of compression enacted by elastic material of the tubular sleeve on the first appendage.

18. A protection apparatus for use with a continuous infusion fluid source and a peripheral fluid line, wherein the peripheral fluid line comprises an internal portion positioned within a vein located in a first appendage of a patient and an external lumen portion positioned on the exterior of the first appendage and configured to interface with a continuous infusion fluid source, the protection apparatus comprising:
a tubular sleeve of a first length configured to concentrically enclose the first appendage and the external lumen portion of the peripheral fluid line,
wherein the tubular sleeve comprises a lumen window configured to enable physical and/or visual access to the external lumen portion of the peripheral fluid line.

19. The protection apparatus of claim 18, wherein the tubular sleeve further comprises a pouch portion located at a distal end of the tubular sleeve, the pouch portion defining a volume configured to contain and secure the continuous infusion fluid source.

20. The protection apparatus of claim 19, wherein the pouch portion is composed of elastic material and is configured to expand to a state in which the continuous infusion fluid source may be inserted into the volume defined by the pouch portion.
